(19) United States
(12) Patent Application Publication
(10) Pub. No.: US 2020/0221824 A1
(43) Pub. Date: Jul. 16, 2020

(54) Foot Orthotic Device

(71) Applicant: Foot Care Store Inc. d.b.a. Dia-Foot, Wellington, FL (US)

(72) Inventor: Robert M. Gaynor, Wellington, FL (US)

(21) Appl. No.: 16/738,157

(22) Filed: Jan. 9, 2020

Related U.S. Application Data
(60) Provisional application No. 62/790,588, filed on Jan. 10, 2019.

(51) Int. Cl. A43B 17/14; A61F 5/14

(52) U.S. Cl. CPC A43B 17/14; A61F 5/14

(57) ABSTRACT

An orthotic device includes: a base layer adapted to extend from a heel counter of a footwear to a toe box of the footwear, the base layer having a heel cup for cradling a heel of a foot; a support layer attached to a top surface of the base layer, the support layer including an anterior portion adapted to underlie and support a metatarsal arch of the foot and a posterior portion adapted to underlie and support the heel; said anterior portion having a first cutout along a medial side and a second cutout along a lateral side to define a protrusion that extends in an anterior direction and is adapted to underlie a second, third, and fourth metatarsals, said protrusion includes a metatarsal raise for supporting the metatarsal arch; and a cushion layer covering said top surface of said base layer and said support layer.

### TECHNICAL FIELD

The present invention relates to foot orthotics, and more specifically, to a foot orthotic insole device that supports and provides comfort to the foot. The foot orthotic insole device further assists in orienting the foot relative to the device for promoting appropriate alignment of foot structures. The present invention also relates to footwear incorporating the foot orthotic insole device.

### BACKGROUND

In general, orthotics are appliances that provide support or a brace for the foot. The use of orthotic devices for both normal street situations and athletic situations has increased because of the need for relief of foot pain and lower extremity pain. Conventional orthotic foot devices can be in the form of removable inserts that are placed inside a shoe. Some shoe inserts extend along only a portion of the foot, for example, from the heel to the ball of the foot. These partial inserts can provide some support, but tend to have a loose fit and often slip inside the shoe. Other shoe inserts extend along the entire length of the foot and are less likely to slip.

However, known orthotic devices are unable to maximize comfort and effectively minimize trauma to the sole of the foot. Many conventional devices are either too “soft” or too “hard” on the foot, thereby making it uncomfortable for the person to wear and greatly increasing the chances that the person stops using the device. These orthotic devices are either custom-molded to an individual's foot or generically made according to an average foot size and structure. The generic versions are manufactured to have a substantially flatter shape and/or are made with overly compressible (overly soft) materials in order to accommodate for differences between the individual's foot and the average foot. As a result, they fail to adequately stabilize the foot and do not uniformly distribute the stress of weight-bearing areas of the foot. Some known devices have been designed to target only certains areas of the foot, such as the cuboid, heel, or phalanges, by merely building up corresponding areas of the device with padding. For example, some orthotic devices simply have a heel cushion on top of the heel, but this area wears down or wears out quickly. As a consequence, the orthotic devices become ineffective in a relatively short period of time.

Other orthotic devices have been designed with rigid or hard materials in order to achieve adequate support. However, such devices fail to conform to the foot and may actually cause pain in the foot. These overly rigid orthotic devices can also inhibit plantar flexion (a flexing of the foot which occurs when the heel is in contact with the ground and the rest of the foot is elevated off of the ground) and dorsiflexion (a flexing of the foot which occurs when the toes are in contact with the ground and the rest of the foot is elevated off of the ground), thereby adversely affecting the user's gait. The user may stop wearing or using the orthotic devices as a result.

Conventional orthotic devices fail to provide effective cushion as well as comprehensive support to the entire foot. Thus, there exists a need for a foot orthotic device that provides cushion and stability for supporting the foot and minimizing arch pain, heel pain, and pain in the metatarsal area, also known as metatarsalgia.

### SUMMARY

The needs set forth herein as well as further and other needs and advantages are addressed by the present embodiments, which illustrate solutions and advantages described below.

It is an object of the present teachings to remedy the above drawbacks and shortcomings associated with prior art orthotic devices and assemblies.

It is an object of the present teachings to solve three major problems people have with their feet: arch pain, heel pain, and metatarsal pain. As such, the purpose of the present teachings is to help people who suffer from arch pain, heel pain, and/or metatarsalgia by providing optimal cushion and support to the foot, and preferably the entire foot.

It is another object to provide relief from discomfort brought about by excessive stress placed on the foot during standing, walking, jogging, running, or other active maneuvers. The present teachings disclose a solution that provides pain relief and support to high-arched feet, normal-arched feet, and flat feet.

These and other objects of the present teachings are achieved by providing a device for use as an orthotic, insole, and/or insert in footwear, wherein the device includes a base layer or sole having a general outline of a foot and a support layer or shell attached on top of the base layer. The support layer extends from the hindfoot to a rear section of the forefoot where the metatarsal bones are located. In particular, the support layer has a posterior portion that is configured to underlie the hindfoot region and an anterior portion that underlies the metatarsal region and terminates short of reaching the metatarsophalangeal joints (i.e., joints between the metatarsal bones of the foot and the proximal phalanges). The support layer is constructed as a polypropylene shell. The base layer, in some embodiments, is made of ethylene-vinyl acetate (EVA) having a hardness of at least 70 A durometer (Shore A scale). For example, the EVA of the base layer is characterized with a 77 A durometer hardness rating. Accordingly, the base layer provides protection for the overall device and keeps the device from breaking down. The support layer or shell is characterized as having a shore hardness greater than that of the base layer, and thus is firmer than the base layer.

The anterior portion of the support layer has a unique configuration which comprises a protrusion or projection that extends forward between a first ray and a fifth ray of a foot. The protrusion has an increased thickness compared to the rest of the support layer to establish a metatarsal raise that supports a second ray, a third ray, and a fourth ray of the foot. Stated differently or in addition to the above, the anterior portion has medial and lateral cutouts at and/or proximate to the first ray and the fifth ray, respectively, so that the anterior portion provides a metatarsal raise that substantially lies underneath the second, third, and fourth rays only. The support layer at the forward end therefore has an outline of a plateau (where the metatarsal raise is positioned) flanked by valleys (where the first ray and fifth ray cutouts are made). The configuration of the cutouts and protrusion of the anterior portion, which forms the metatarsal raise, is especially adapted to help alleviate pain in the metatarsals.

The posterior portion of the support layer has an outline resembling generally the shape of a heel area of the base layer. Another unique feature of the support layer is that the posterior portion includes a hole or opening in the heel area. A heel cushion insert is disposed within the opening and attached to the top surface of the base layer. The shape of the cushion insert corresponds to the shape of the opening so that they mate with each other. The heel opening and the cushion insert positioned therein are adapted to help alleviate pain in the heel.

A middle portion of the support layer is curved to support the longitudinal arches of the foot. A lateral arch support is integrated into a lateral (outer) side of the support layer, while a medial arch support is integrated into a medial (inner) side of the support layer. Both the medial arch support and the lateral arch support are defined by curved areas of the support layer. However, the medial arch support is curved upward greater than the lateral arch support. Both support elements work together to provide stability to the foot and to relieve pain in the arches.

The device according to the present teachings further comprises an intermediate layer having the general outline of the foot and being disposed on top of the support and base layers. More specifically, the intermediate layer adheres to the support layer in the posterior and middle portions, as well as the metatarsal section of the anterior portion. Since the support layer terminates at or before the metatarsophalangeal joints, the intermediate layer is attached to the base layer in the phalange section of the forefoot. The intermediate layer is made of soft, heat-moldable EVA to provide cushion to the entire foot. The intermediate layer is configured to conform to the contours and curvatures of the foot upon reaction to heat.

In some embodiments, the device according to the present teachings also includes a foam layer being disposed on top of the intermediate layer. The foam layer provides additional cushion to the entire foot. The foam layer may be made of regular polyurethane foam, or more preferably memory foam.

The device according to the present teachings further includes a top layer made of mesh, and more specifically, anti-microbial mesh. However, in other embodiments, the top layer may be a thin layer of leather. Like the intermediate layer, the top layer has a general outline of the foot.

Other features and aspects of the present invention will become apparent from the following detailed description, taken in conjunction with the accompanying drawings, which illustrate by way of example the features in accordance with embodiments of the invention. The summary is not intended to limit the scope of the invention, which is defined solely by the claims attached thereto.

### BRIEF DESCRIPTION OF THE DRAWINGS

FIG. 1 is perspective view of the foot orthotic device according to the present teachings.

FIG. 2 is an exploded view in perspective of the orthotic device of FIG. 1.

FIG. 3 is a side view of the orthotic device of FIG. 1.

FIG. 4 is a side view of the orthotic device of FIG. 1, which is opposite to the side shown in FIG. 3.

FIG. 5 is a top view of the orthotic device of FIG. 1.

FIG. 6 is a bottom view of the orthotic device of FIG. 1.

It should be understood that throughout the drawings, corresponding reference numerals indicate like or corresponding parts and/or features.

### DETAILED DESCRIPTION

The present teachings are described more fully hereinafter with reference to the accompanying drawings, in which the present embodiments are shown. The following description illustrates the present teachings by way of example, not by way of limitation of the principles of the present teachings.

It is to be understood that the present teachings are not limited to the specific features shown and described, since the devices herein disclosed comprise preferred forms of putting the present teachings into effect.

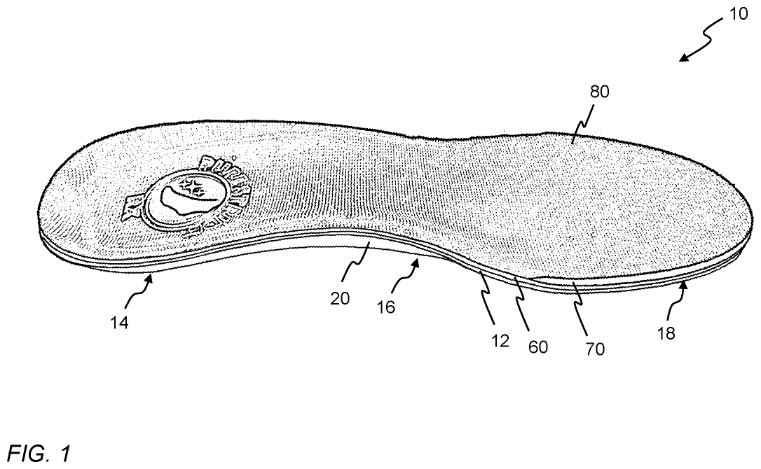

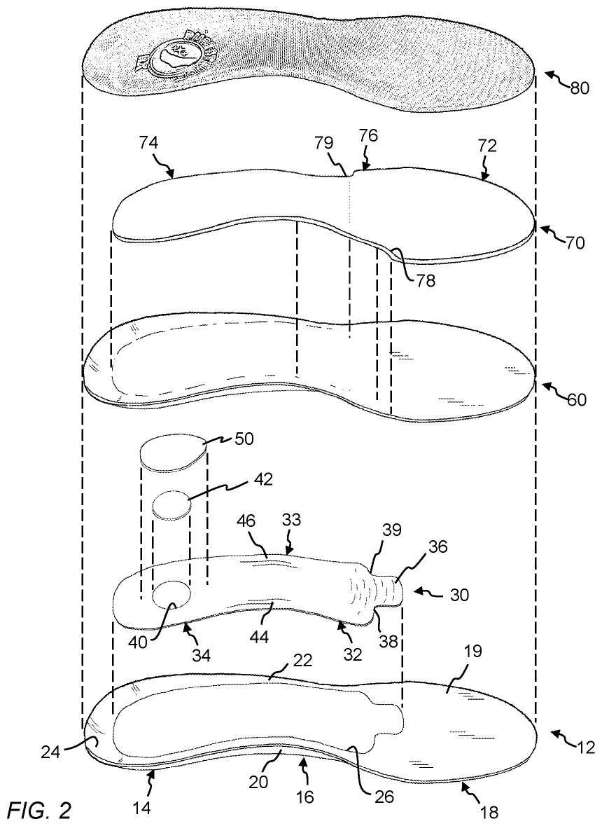

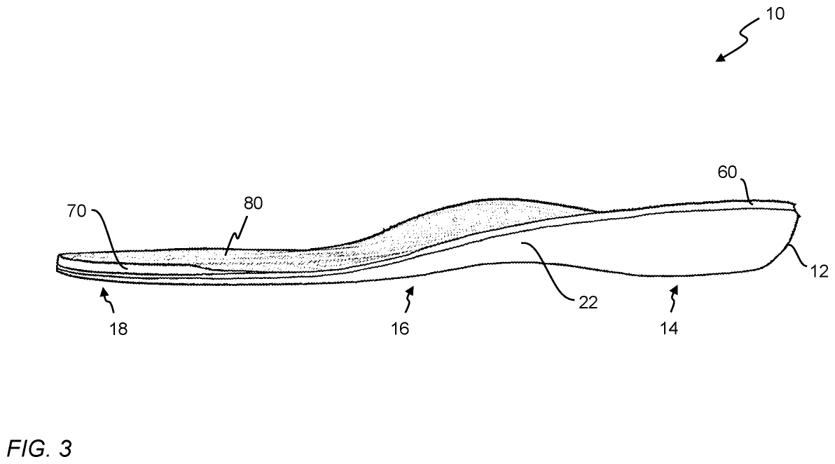

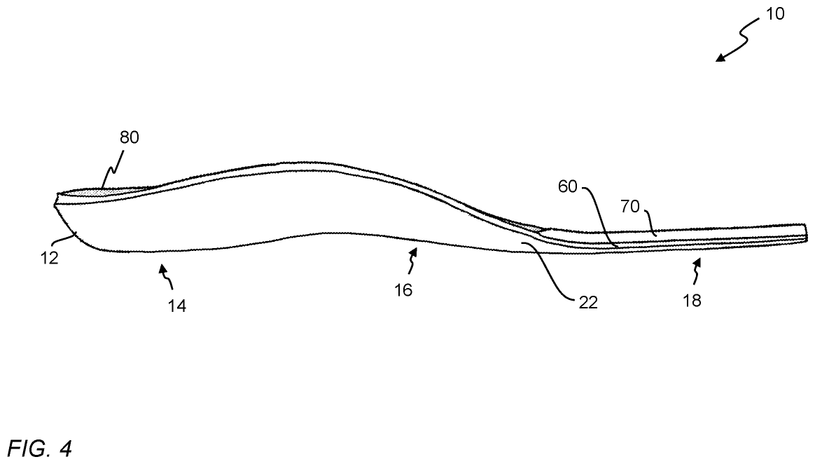

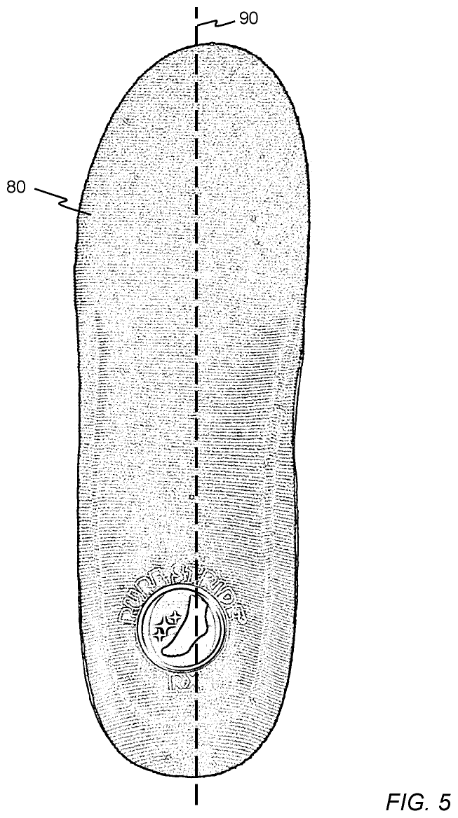

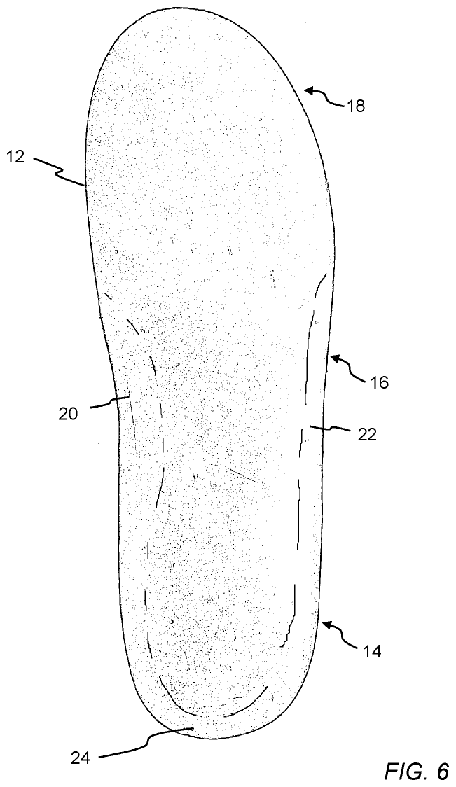

Referring to FIGS. 1-2, an orthotic device 10 according to the present teachings is shown. The orthotic device 10 is designed to fit within, be placed into or on top of any type of footwear, or be integrated as a part of the footwear. The device comprises a sole or base layer 12 and a shell or support layer 30 attached to a top surface of the base layer 12. The base layer 12, as shown in FIG. 6, has the general outline of a foot, with a hindfoot section 14, a midfoot section 16, forefoot section 18. The base layer is adapted to extend from a heel counter of the shoe to a toe box of the shoe. The forefoot section 18 provides a planar surface 19, while the hindfoot and midfoot sections include various curvatures and contouring to resemble the arch of a foot. In particular, the base layer 12 curves slightly upwards from the hindfoot section towards a peak in the midfoot section before curving down and flattening out at the forefoot section. The arch structure of the midfoot section 16 is also defined by a medial (inner) side that curves upwards from a center line 90 (FIG. 5) of the orthotic device towards the medial edge. A lateral (outer) side 22 of the midfoot section 16 also curves slightly upwards from the center line 90 to the lateral edge. As shown through a comparison of FIGS. 3 and 4, the curvature of the medial side is greater than the curvature of the lateral side. The medial side 20 is structurally designed to support at least the medial area of the plantar fascia, including the navicular and medial cuneiform bones. In contrast, the lateral side 22 is structurally designed to support at least the lateral area of the plantar fascia, including the cuboid bone. Since more strain is present on the medial area of the plantar aspect of the midfoot, where the longitudinal arch is more pronounced, the medial side 20 has a greater curve height than the lateral side 22 of the base layer.

The hindfoot section 14 of the base layer comprises a cup-shaped backing 24 that is integrally formed with and connected to the medial and lateral sides 20, 22. The cup-shaped backing is a semi-circular raised lip that extends along the periphery of the hindfoot section 14. As a result, the base layer via the cup-shaped backing 24 is adapted to cradle the heel of the person's foot, thereby bracing, supporting, and providing protection to the heel.

The base layer 12 as a whole with the planar surface 19, medial and laterals sides 20, 22, and the cup-shaped backing 24 may be formed as a monolithic structure. The base layer, for example, may be constructed by a molding process, such as but not limited to injection molding, to give the base layer its defined curvatures and contouring. In other embodiments, multiple components are constructed separately and subsequently joined to each other to form the entire base layer. The base layer 12 is made of a resilient, shock-absorbing material that has sufficient rigidity to control foot motion, such as Ethylene-vinyl acetate (EVA) having a hardness of at least 70 A durometer (Shore A scale). In preferred embodiments, the base layer 12 is made of EVA having a hardness between 70 A and 90 A durometer (inclusive), and more preferably, at or approximately 77 A durometer. The term “approximately” with respect to hardness means ±1 A of the indicated value, unless otherwise indicated. EVA with a shore A hardness of 70 or greater provides the necessary resilience and shock absorption to withstand the impacts experienced by the orthotic device during standing, walking, jogging, running, and any other athletic movements. Accordingly, the base layer 12 provides protection for the overall device and keeps it from breaking down.

The shell or support layer 30, which is attached to the base layer, extends from the hindfoot section 14 up to or approximately to where the forefoot section 18 begins. As shown in FIG. 2, the support layer has an extension in a longitudinal direction (e.g., along centerline 90), the extension being curved. That is, the entire support layer is not flat. Instead, starting from an anterior portion 32, the bottom surface of the support layer 30 curves upward to a peak in a middle region 33 and then downward towards a posterior portion 34. The anterior portion 32 of the support layer is configured to be positioned underneath and thus provide support and stability to the metatarsals and metatarsal arch. In some embodiments, the anterior portion 32 underlies the metatarsal region and ends at or short of reaching the metatarsophalangeal joints (i.e., joints between the metatarsal bones of the foot and the proximal phalanges). That is, the support layer 30 has an anterior portion that terminates at or proximate to a longitudinal point where the planar surface 19 of the support layer 12 begins. The posterior portion 34 of the support layer 30 is positioned on the hindfoot section 14 of the base layer 12. The posterior portion is configured so that it ends at or proximate to a longitudinal point where the cup-shaped backing 24 begins. Stated differently, the posterior portion 34 does not extend up onto the raised lip of the cup-shaped backing 24.

One of the main functions of the support layer is to provide support and stability to the foot, especially to the heel, arch, and metatarsal region of the foot. With this in mind, the support layer 30 has a shore hardness greater than that of the base layer 12, and thus is more rigid than the base layer 12. For example, but limited thereto, the support layer may have a hardness of at least 80 A durometer (Shore A scale) or at least 30 D durometer (Shore D scale). A polymer-based material is used in making the support layer, for example polypropylene. The support layer 30 is designed and constructed as a monolithic shell. The support layer, for example, may be constructed by a molding process, such as but not limited to injection molding, to give the base layer its defined curvatures and contouring. The polymer-based (e.g., polypropylene) material is configured to retain the shapes, thicknesses, and resiliency of the support layer for at least the life of the orthotic foot device.

The support layer 30 serves as the central component of the entire orthotic device and is especially adapted to help relieve heel pain, arch pain, and metatarsal pain. As such, all other components of the orthotic device 10 are linked to the support layer 30. In particular, the support layer comprises a unique configuration that addresses the three major types of foot pain experienced by people. As shown in FIG. 2, the support layer 30 has a heel-to-ball length, and thus is configured to extend from the heel to the ball of the foot. The anterior portion 32 of the support layer includes a protrusion or projection 36 that extends forward in the anterior direction between a first ray and a fifth ray of the foot. The protrusion 36 is specifically designed to be a metatarsal raise providing support to the foot at the second ray, third ray, and fourth ray (i.e., second, third, and fourth metatarsals). In particular, the protrusion 36 itself has a thickness(es) that is greater than the rest of the support layer 30, as illustrated by the contour lines in FIG. 2. For example, the thickness of the protrusion itself can vary and is configured to follow the outline of the metatarsal arch, thereby providing optimal support to the plantar fascia ligament at this part of the foot. A gradual slope or tapering in the posterior direction is present as the increased thickness of the protrusion transitions to the thickness of the remaining portion of the support layer. In addition, the thickness of the protrusion 36 may gradually increase from medial (inner) and lateral (outer) edges of the protrusion towards the center line 90. The shape and configuration of the protrusion can be described in a manner alternative to or in addition to the above. That is, the anterior portion 32 includes a medial (inner) cutout or recess 38 at and/or proximate to the first ray, as well as a lateral (outer) cutout or recess 39 at and/or proximate to the fifth ray. The medial and lateral cutouts 38, 39 help with metatarsal pain. The medial and lateral cutouts provide concave portions or corners that define the borders of the protrusion 36 and thus establish the metatarsal raise to be situated predominantly underneath the second, third, and fourth rays (more specifically, second, third, and fourth metatarsals). Due to the cutouts, the anterior end of the support layer has an outline of a plateau (where the protrusion is positioned) flanked by valleys (where the medial and lateral cutouts are made). In some embodiments, the corners formed by the medial and lateral cutouts may be sharp. In other embodiments, rounded corners are provided at the protrusion 36 and cutouts 38, 39. Alternatively, the cutouts 38, 39 may provide linear sloped edges such that the protrusion has a trapezoidal shape.

As shown in FIG. 2, the posterior portion 34 of the support layer 30 has an outline resembling generally the shape of a heel area of the base layer 12. Another unique feature of the support layer 30 is that there is hole or opening 40 in the posterior portion 34. The opening 40 is positioned substantially in the center of the posterior portion to be aligned with the heel bone (calcaneus) of the foot. The shape of the opening 40 may differ in various embodiments, but preferably it is circular or oval. A heel cushion insert 42 is disposed within the opening 40 and attached to the top surface of the base layer 12. The heel cushion insert 42 has a substantially planar structure with a thickness substantially equal to the thickness of the posterior portion 34 of the support layer 30. The shape of the cushion insert 42 corresponds to the shape of the opening (e.g., circular) so that they mate with each other. As a result, the posterior portion 34 and the heel cushion insert 42 cooperate to provide a smooth surface at their interface. Alternatively, the heel cushion insert may be slightly thicker than the posterior portion 34 at the edge of the opening 40. In some embodiments, the heel cushion insert 42 is made of open-cell foam, such as open-cell urethane foam. In preferred embodiments, the heel cushion insert is made of Poron®. The use of open-cell foam, like Poron®, enables the heel cushion insert to maintain its original form, provides durability, and adds long-lasting performance and comfort to the orthotic device without adding any weight and bulk. In other embodiments, the heel cushion insert 42 may be made of rebound foam, EVA, or any other type of cushioning product. The heel opening 40 and the cushion insert 42 provide a high degree of shock absorption and are thus adapted to help alleviate heel pain.

A middle portion 33 of the support layer 30 is situated between the anterior and posterior portions 32, 34. The middle portion 33 is curved to support to the arches of the foot in the longitudinal direction. Within the middle portion 33, a lateral arch support 46 is integrated into a lateral (outer) side of the support layer, while a medial arch support 44 is integrated into a medial (inner) side of the support layer. The medial arch support and the lateral arch support are constituted as curved areas of the support layer 30. For example, the medial arch support 44 comprises a curvature or curved structure, as illustrated by contour lines in FIG. 2, that starts from the posterior portion 34, at or proximate to the calcaneus and the talus, and extends along the navicular and cuneiforms to the first, second, and/or third metatarsals. The lateral arch support 46 comprises a curvature or curved structure, as illustrated by contour lines, that starts from the posterior portion 34, at or proximate to the calcaneus and extends along the cuboid to fourth and/or fifth metatarsals. Since the lateral arch is the flatter of the two longitudinal arches of the foot, the lateral arch support 46 is configured to be curved upwards (in a superior direction) less than the medial arch support 44. In other words, since the medial arch is the higher of the two longitudinal arches, the medial arch support 44 comprises a curve height greater than that of the lateral arch support 46. Accordingly, the longitudinal arch supports 44, 46 provide support and stability to the plantar fascia ligament. It is noted that in some embodiments, the support layer 30 only has one of the longitudinal arch supports—either the medial arch support 44 or the lateral arch support 46. In yet other embodiments, the support layer 30 may have no longitudinal arch supports.

Referring back to the base layer 12, this component of the orthotic device may comprise a depression or recessed area 26 that extends from the hindfoot section 14 towards the forefoot section 18. More specifically, the depression 26 has the same or substantially the same shape and size as the outline of the support layer 30. As such, when the support layer 30 is attached to the base layer 12, the support layer is in mating relationship with the depression 26 such that a smooth surface is formed between the two layers at their interface. Some embodiments of the orthotic device may include the base layer without the depression.

As an optional component, the orthotic device 10 may include a heel pad 50 that is disposed over the heel cushion insert 42 and the immediate area surrounding the opening 40. The heel pad therefore has a larger size than the heel cushion insert 42. As shown in FIG. 2, the heel pad has a substantially planar form. In some embodiments, the shape of the heel pad 42 is circular. In other embodiments, the shape of the heel pad is oval or egg-shaped. The heel pad 50 provides additional support and cushion for the heel. Furthermore, the heel pad provides protection for the heel cushion insert, thereby minimizing wear and deterioration of the heel cushion insert. In some embodiments, soft EVA (ethylene-vinyl acetate) is used in construction of the heel pad. With respect to the level of softness, the heel pad may be characterized as having Shore A durometer rating of 50 A or less. In some embodiments, the heel pad may have a Shore A durometer rating of 30 A or less. In yet other embodiments, the heel pad may be constructed to have a Shore 00 rating of 70 or less.

The orthotic device 10 according to the present teachings further comprises an intermediate layer 60, which is disposed on top of the support layer 30 and heel cushion insert 42, and if present, also the heel pad 50. The intermediate layer has the general outline of the foot. When the intermediate layer is placed into position, it conforms to the shape of the combined structure of the base layer 12, support layer 30, and heel cushion insert 42. More specifically, the intermediate layer adheres to the support layer 30. Also, since the support layer 30 terminates at or before the metatarsophalangeal joints, the intermediate layer 60 is attached directly to the base layer 12 in an area of the phalanges at the forefoot section 18. In some embodiments, the intermediate layer 60 is made of soft, heat-moldable EVA to provide cushion and comfort to the entire foot. The intermediate layer 60 is therefore configured to conform to the contours and curvatures of the support and base layers. In addition, by way of the EVA material, the intermediate layer is configured to heat mold to the foot as the person wears the orthotic device. Like other layers of the orthotic device, the intermediate layer may be constructed by a molding process, such as but not limited to injection molding. In some embodiments, the heel pad 50 and the intermediate layer 60 are constructed together as a single monolithic structure. This can be accomplished through the molding process.

To further increase cushion and comfort, an optional foam layer 70 may be added to the orthotic device. The foam layer 70 may be disposed on top of the intermediate layer 60. The foam layer may be made of regular polyurethane foam, or more preferably memory foam. As shown in FIG. 2, the foam layer has the same or substantially the same outline as the base layer 12 and/or the intermediate layer 60 at an anterior portion 72. However, a posterior portion 74 and middle portion 76 of the foam layer slightly diverge in shape from the base layer 12 and/or the intermediate layer 60. In particular, the posterior portion 74 ends short of the cup-shaped backing 24 and thus does not overlie this part of the base layer. The foam layer also includes a recess 78 along its medial side in the middle portion 76, such that the medial edge of the foam layer does not align with corresponding edges of the base layer 12 and intermediate layer 60. Similarly, the foam layer includes a recess 79 along its lateral side, such that the lateral edge of the foam layer does not align with corresponding edges of the base layer 12 and intermediate layer 60.

Referring to FIG. 5, the orthotic device 10 also comprises a mesh 80 disposed over the intermediate layer 60 and, if present, the foam layer 70. The mesh 80 serves as the top surface of the orthotic device. In a preferred embodiment, the mesh 80 is an antimicrobial mesh having one or more antimicrobial agents that are configured to kill or slow the spread of microorganisms, such as bacteria. This helps to prevent microbes from getting into the person's foot. The mesh may be made of fabric. As an alternative to the mesh 80, a thin layer of leather may be disposed on top of the intermediate layer 60. The mesh 80 has a thickness predetermined for its particular use. As shown in FIGS. 1-3, the mesh 80 has a thickness substantially less than the respective thicknesses of the intermediate layer 60 and foam layer 70.

The various layers described above (e.g., base layer 12, support layer 30, heel cushion insert 42, heel pad 50, intermediate layer 60, foam layer 70, antimicrobial mesh 80) are attached to each other by glue or an adhesive. In other embodiments, stitching is used to join each of the layers of the orthotic device. In yet other embodiments, a combination of adhesive and stitching may be used to firmly attach the layers to one another.

The orthotic device according to the present teachings functions as being firm due to the polypropylene shell of the support layer 30, yet comfortable to wear due to the soft EVA intermediate layer 60 and, if present, the memory foam layer 70. The foam layer 70 is optional as it adds additional cushioning to the orthotic device, but the device remains functional without the foam layer 70. The mesh 80 is also optional and could be replaced with any other type of material. With the above exemplary configuration of layers, the orthotic device 10 places the foot is in proper alignment, thereby facilitating uniform balance and distribution of weight over the entire foot without producing undue stress on any part thereof. It is ensured that the orthotic device of the present teachings provides the necessary support and cushion to alleviate heel, arch, and metatarsal pain without adversely affecting the person's gait.

It is noted that the orthotic device 10 can be modified by adding additional components to help with other problems that a person may be having. For example, a heel lift can be added to help a person who has a limb length issue or Achilles tendinitis. Sticky pads can also be added to help a person with neuromas.

The present teachings also provide for a method of making the foot orthotic device 10. The orthotic device 10 may be made in a factory setting. The support layer 30 is first molded from polypropylene for example and cut out to specification to include the protrusion 36 and the medial and lateral cutouts 38, 39. A material such as EVA having at least 70A durometer rating is provided in blocks. These blocks are heated, placed into a mold and shaped to be the base layer 12. The support layer 30 is subsequently glued and/or stitched onto the base layer. The heel cushion insert 42 is molded into shape using Poron®, and if necessary, cut to specification so that it fits in mating relationship with the opening 40 in the support layer 30. The heel cushion insert 42 is glued to the base layer within the opening 40. The intermediate layer 60 is molded and/or cut into shape using soft, heat-moldable EVA. The foam layer 70 is molded and/or cut to specification using memory foam material. A sheet of fabric mesh material (e.g., antimicrobial mesh) is cut to specification to form the mesh 80. The intermediate layer 60, foam layer 70, and mesh 80 are joined with each other and to the base layer 12 and the support layer 30 by way of glue and/or stitching. For example, the layers 60, 70, and 80 are glued and pressed onto the base and support layers. A die cut mold may be used to cut the shape and size of the orthotic. In some embodiments of the orthotic device 10, there is no foam layer 70. Accordingly, the method of making these particular embodiments would not involve the step of molding a foam layer.

The present teachings also provide for a method of using the orthotic device. A person would remove the inserts or insoles already present in the shoes. The person would then insert one orthotic device 10 into each shoe. The person would wear the orthotic device for a few hours (e.g., 3-6 hours) and if pain develops, the person would remove the orthotic device from each shoe. The person would then wear the orthotic devices 10 again the next day until the person's feet are accustomed to wearing the orthotics for a full day. The orthotic device 10 is cleaned with warm soap water.

It should be understood to a person of ordinary skill in the art that different configurations of the foot orthotic device are possible. For example, the arrangement and order of the support layer, cushion layer, foam layer, heel cushion insert, and heel pad may differ from those shown in the Figures without departing from the scope and spirit of the present teachings. The components included in the orthotic device may also differ from that shown in the Figures without departing from the scope and spirit of the present teachings.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of the appended claims and their legal equivalents, as understood by those of skill in the art relying upon the disclosure in this specification and the attached drawings.

What is claimed is:

1. An orthotic device comprising:
a base layer adapted to extend from a heel counter of a footwear to a toe box of the footwear, said base layer having a heel cup for cradling a heel of a foot;
a support layer attached to a top surface of said base layer, said support layer includes an anterior portion adapted to underlie and support a metatarsal arch of the foot and a posterior portion adapted to underlie and support the heel;
said anterior portion having a first cutout along a medial side and a second cutout along a lateral side to define a protrusion that extends in an anterior direction and is adapted to underlie a second, third, and fourth metatarsals, said protrusion includes a metatarsal raise for supporting the metatarsal arch; and
a cushion layer covering said top surface of said base layer and said support layer.

2. The orthotic device of claim 1, wherein said posterior portion of said support layer includes a hole aligned with a midline extending longitudinally of said support layer, said hole being adapted to underlie the heel.

3. The orthotic device of claim 2, further comprising a heel cushion inserted within said opening and attached to said base layer.

4. The orthotic device of claim 3, wherein said heel cushion is made of an open-cell foam material.

5. The orthotic device of claim 1, wherein said support layer is a shell having a hardness greater than that of said base layer with respect to a durometer shore scale.

6. The orthotic device of claim 1, wherein said support layer forms a shell made of polypropylene.

7. The orthotic device of claim 1, wherein said base layer comprises ethylene vinyle acetate (EVA).

8. The orthotic device of claim 7, wherein said base layer has a shore hardness of at least 70 A.

9. The orthotic device of claim 8, wherein said base layer has a shore hardness equal to or approximately equal to 77 A.

10. The orthotic device of claim 1, wherein said cushion layer is made of a heat-moldable material that is adapted to conform to the foot upon reaction to heat.

11. The orthotic device of claim 1, wherein said cushion layer comprises ethylene vinyle acetate (EVA).

12. The orthotic device of claim 11, wherein said cushion layer has a shore hardness equal to or less than 50 A.

13. The orthotic device of claim 1, wherein said support layer includes a middle portion situated between said anterior and posterior portions, said middle portion includes a curved contour along the medial side of said support layer for supporting a medial arch and/or a curved contour along the lateral side of said support layer for supporting a lateral arch.

14. The orthotic device of claim 1, further comprising a foam layer disposed over said cushion layer, said foam layer providing cushion.

15. The orthotic device of claim 1, further comprising a mesh fabric disposed over said cushion layer.

16. The orthotic device of claim 15, wherein said mesh fabric includes an anti-microbial additive, agent, or coating.

17. An orthotic device comprising:
a base layer adapted to extend from a heel counter of a footwear to a toe box of the footwear, a hindfoot section of said base layer having a heel cup for cradling a heel of a foot, said base layer having a top surface with a recess that extends from the hindfoot section towards a forefoot section;
a support layer inserted into said recess and attached to said base layer, said support layer includes an anterior portion adapted to underlie and support a metatarsal arch of the foot and a posterior portion adapted to underlie and support the heel;
said anterior portion having a first cutout along a medial side and a second cutout along a lateral side to define a protrusion that extends in an anterior direction and is adapted to underlie a second, third, and fourth metatarsals, said protrusion includes a metatarsal raise for supporting the metatarsal arch; and
a cushion layer covering said top surface of said base layer and said support layer, said cushion layer being adapted to extend from the heel counter to the toe box.

18. The orthotic device of claim 17, wherein said support layer mates with said recess to provide a smooth surface at the interface between said base layer and said support layer.

19. The orthotic device of claim 17, further comprising a heel cushion;
wherein said posterior portion of said support layer includes a hole aligned with a midline extending longitudinally of said support layer, said heel cushion being disposed within said opening and being adapted to underlie the heel.

20. The orthotic device of claim 17, wherein said heel cushion is made of an open-cell foam material.

21. The orthotic device of claim 17, wherein said base layer comprises ethylene vinyle acetate (EVA) and has a shore hardness of at least 70 A; and
wherein said cushion layer comprises ethylene vinyle acetate (EVA) and has a shore hardness equal to or less than 50 A.
